(19) United States
(12) Patent Application Publication
(10) Pub. No.: US 2025/0114333 A1
(43) Pub. Date: Apr. 10, 2025

(54) METHODS AND COMPOSITIONS FOR THE TREATMENT OF EPILEPSY

(71) Applicant: ARIZONA BOARD OF REGENTS ON BEHALF OF THE UNIVERSITY OF ARIZONA, Tucson, AZ (US)

(72) Inventors: Michael F. Hammer, Tucson, AZ (US); Erfan Bahramnejad, Tucson, AZ (US)

(21) Appl. No.: 18/952,827

(22) Filed: Nov. 19, 2024

Related U.S. Application Data
(63) Continuation of application No. PCT/US23/67230, filed on May 19, 2023.
(60) Provisional application No. 63/343,721, filed on May 19, 2022.

(51) Int. Cl. A61K 31/4184; A61P 25/08

(52) U.S. Cl. CPC A61K 31/4184; A61P 25/08

(57) ABSTRACT

Epilepsy is one of the most common neurological disorders. However, there are currently no drugs available to prevent and/or reduce the development of said disorder. Thus, compositions and methods described herein prevent and/or arrest the development of epilepsy. Specifically, angiotensin receptor blocker (ARB), such as candesartan, is described to treat epileptic patients and those at risk for developing epilepsy.

### CROSS-REFERENCES TO RELATED APPLICATIONS

This application is a continuation and claims benefit of PCT Application No. PCT/US2023/067230 filed May 19, 2023, which claims benefit of U.S. Provisional Application No. 63/343,721 filed May 19, 2022, the specification of which is incorporated herein in its entirety by reference.

### FIELD OF THE INVENTION

The present invention features compositions and methods for the treatment of epileptic disorders.

### BACKGROUND OF THE INVENTION

Approximately 1 in 26 people will develop epilepsy at some point. Arizona has several higher-risk populations, including seniors, Native Americans, Latino communities, children, active military members, and veterans. Alarmingly, nearly half of the more than 77,000 Arizonans treated for active epilepsy continue to have seizures.

Currently, no drugs prevent or reverse the development of epilepsy, one of the most common neurological disorders. Given the high cost of bringing new medicines to market, drug repurposing offers the potential for significant savings in the time and cost of drug development. The present invention provides mechanistic insights into pathways that underlie epileptogenesis and determines which pathway effects are reversed by administering an angiotensin receptor blocker (ARB) with efficacy in treating epilepsy.

### BRIEF SUMMARY OF THE INVENTION

It is an objective of the present invention to provide compositions and methods that allow for mitigating the process of epileptogenesis, as specified in the independent claims. Embodiments of the invention are given in the dependent claims. Embodiments of the present invention can be freely combined with each other if they are not mutually exclusive.

Following the primary insult, whether mechanical or genetic, is a secondary injury cascade that includes a myriad of neuropathological processes, such as oxidative stress, neuroinflammation, astrocytosis, and disruption of the blood-brain barrier (BBB). The present invention demonstrates the therapeutic potential of angiotensin receptor blockers (ARB) in countering the secondary injury cascade.

In some embodiments, the present invention features a method of preventing or treating an epileptic condition (e.g., epilepsy) in a patient in need thereof. For example, the method may comprise administering a therapeutically effective amount of an angiotensin receptor blocker (ARB) to the patient.

In other embodiments, the present invention may also feature a method of protecting the blood-brain barrier (BBB) in a subject in need thereof. The method comprises administering a therapeutically effective amount of an angiotensin receptor blocker (ARB) to the subject.

In some embodiments, the present invention may also feature a method of preventing a seizure in a subject in need thereof, the method comprising administering a therapeutically effective amount of an angiotensin receptor blocker (ARB) to the patient.

In other embodiments, the present invention features a composition comprising an angiotensin receptor blocker (ARB) for use in a method of treating an epileptic condition (e.g., epilepsy) in a patient in need thereof.

One of the unique and inventive technical features of the present invention is using an angiotensin receptor blocker (ARB; e.g., candesartan (CAN)) to prevent or slow the progression of epileptogenesis. Without wishing to limit the invention to any theory or mechanism, it is believed that the technical feature of the present invention advantageously provides for the treatment of epileptic conditions through the protection of the blood-brain barrier. None of the presently known prior references or work has the unique, inventive technical feature of the present invention.

Furthermore, the prior references teach away from the present invention. For example, angiotensin receptor blockers (ARBs) are currently indicated in hypertension, not epilepsy. Additionally, ARBs have no known epilepsy-related targets (e.g., ion channels or neurotransmitter receptors).

Furthermore, the inventive technical features of the present invention contributed to a surprising result. For example, in a mouse model of severe pediatric epilepsy, treatment of mice before seizures led to statistically significant delays in seizure onset, while treatment of mice at the time of seizure onset reduced seizure frequency and increased survival. These effects were robust in juvenile mice, as well as adult females and males.

Any feature or combination of features described herein is included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows ARB-dosing regimes for juvenile and adult mice. Gray bars indicate the approximate start and end dosing times based on variable seizure onset and survival time, respectively. Black bars indicate hard start dosing times and/or certain periods of dosing based on a range of typical survival times.

FIG. 2 shows B6 Adult Male Survival and Seizure Frequency in untreated versus ARB-treated Late or ARB-treated Early Groups.

FIG. 3 shows a B6 Adult Male Gap (e.g., between seizures) Analysis in Untreated versus ARB-treated Late or ARB-Treated Early Groups.

FIG. 4 shows male lifetime seizures in untreated versus ARB-treated (i.e., a treated Late Group).

FIG. 5 shows B6 Adult Male Age at Onset and Mode of Death in Untreated versus ARB-Treated Late or ARB-Treated Early Groups; CSE=convulsive status epilepticus, SUD=SUDeP, DEC=decompensated.

FIG. 6 shows B6 Adult Male Survival in untreated versus ARB-Treated.

FIG. 7 shows B6 Juveniles in untreated versus ARB-or Phenytoin (PHT)-Treated Group.

FIG. 8 shows B6 Juvenile Survival in untreated versus ARB-or Phenytoin Treated Groups.

FIG. 9 shows C3H Male and Female Adults Survival in Untreated versus ARB-Treated Late Groups.

FIG. 10 shows that the results described herein are robust to strain, sex, and life stage.

FIG. 11 shows the extent of blood-brain-barrier disruption (BBBD) in untreated heterozygous (D/+) pre-seizure mice is elevated in both sexes relative to wildtype controls, although to a lesser extent in females. Treated pre-seizure mice show reduced BBBD, with permeability returning to baseline. Post-seizure mice of both sexes show a large increase in BBBD; however, permeability returns to near baseline in treated mice of both sexes.

FIGS. 12A and 12B show the effect of seizures on gene expression across the genome (transcriptome) in untreated homozygous D/D juvenile mice and changes in gene expression in response to treatment. FIG. 12A shows treated juveniles have fewer dysregulated genes than wildtype controls. FIG. 12B shows a pathway analysis indicates that untreated post-seizure juveniles activate a number of pathways involved in neuroinflammation, astrocytosis (fibrosis), cellular remodeling, and mitochondrial dysfunction. These pathways are returned to physiological baseline in treated juveniles, while mitochondrial function is enhanced.

Referring to the figures, results of statistical summaries are expressed as mean±SD. Kaplan-Meier survival curves were used to test for differences in survival. Unpaired t-tests were used to test for differences in survival, and chi-square tests were applied to test for differences modes of deaths. In cases where groups did not have the same variance, two-sample t-tests were performed.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult, children, and newborn subjects, as well as fetuses, whether male or female, are intended to be included. In one embodiment, the subject is a mammal (e.g., a human) having a disease, disorder, or condition described herein. In another embodiment, the subject is a mammal (e.g., a human) at risk of developing a disease, disorder, or condition described herein. A “patient” is a subject afflicted with a disease or disorder. In certain instances, the term patient refers to a human.

The terms “treating” or “treatment” refers to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., an angiotensin receptor blocker (ARB)) that is sufficient to reduce and/or ameliorate the severity and/or duration of a given disease, disorder, or condition and/or a symptom related thereto. This term also encompasses an amount necessary for the reduction or amelioration of the advancement or progression of a given disease (e.g., epileptic condition), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of an angiotensin receptor blocker (ARB) herein is an amount sufficient to provide a therapeutic benefit in the treatment or management of an epileptic condition or to delay or minimize one or more symptoms associated with the epileptic conditions. A therapeutically effective amount of an angiotensin receptor blocker (ARB) described herein means an amount of therapeutic agent, alone or in combination with other therapies, which provides a therapeutic benefit in the treatment or management of epileptic conditions. The term “therapeutically effective amount” can encompass an amount that improves overall therapy, reduces or avoids symptoms or causes, or enhances the therapeutic efficacy of another therapeutic agent.

The terms “administering,” and “administration” refer to methods of providing a pharmaceutical preparation to a subject. Such methods are well known to those skilled in the art and include, but are not limited to, administering the compositions intranasally, parenterally (e.g., intravenously and subcutaneously), by intramuscular injection, by intraperitoneal injection, intrathecally, transdermally, extracorporeally, topically or the like.

As used herein, “epileptogenesis” refers to the process between an initial injury (latent phase), the development of an epileptic condition (acute phase), and the progression of epilepsy after it is established (chronic phase).

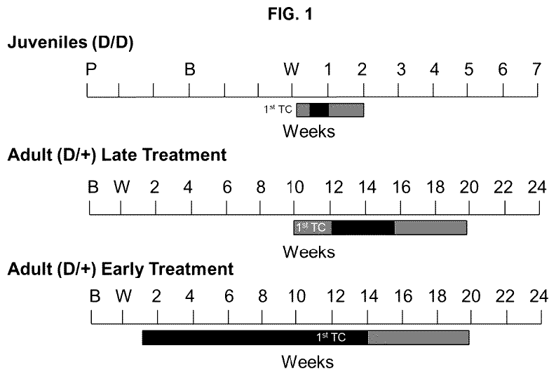

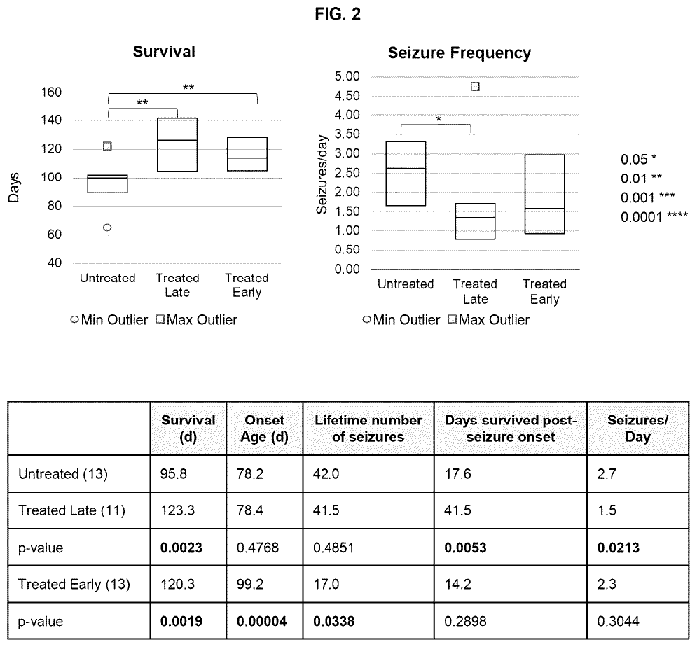

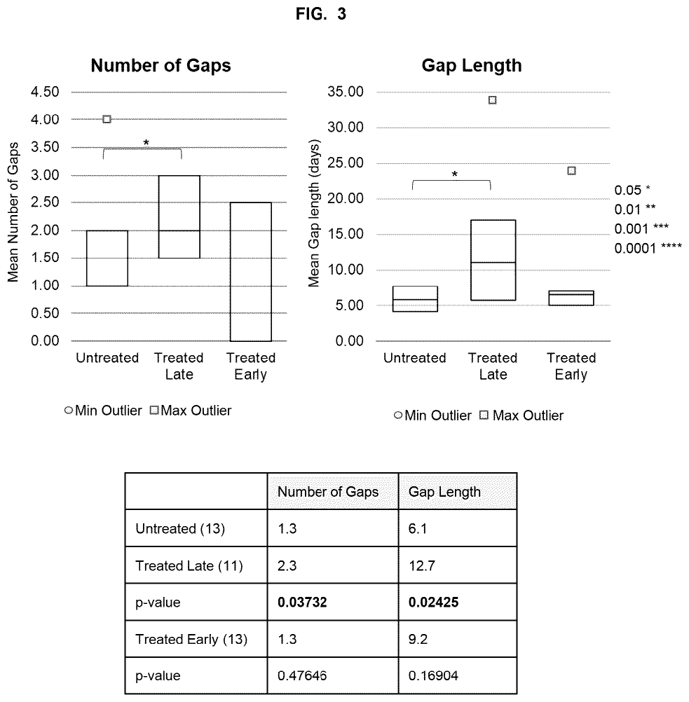

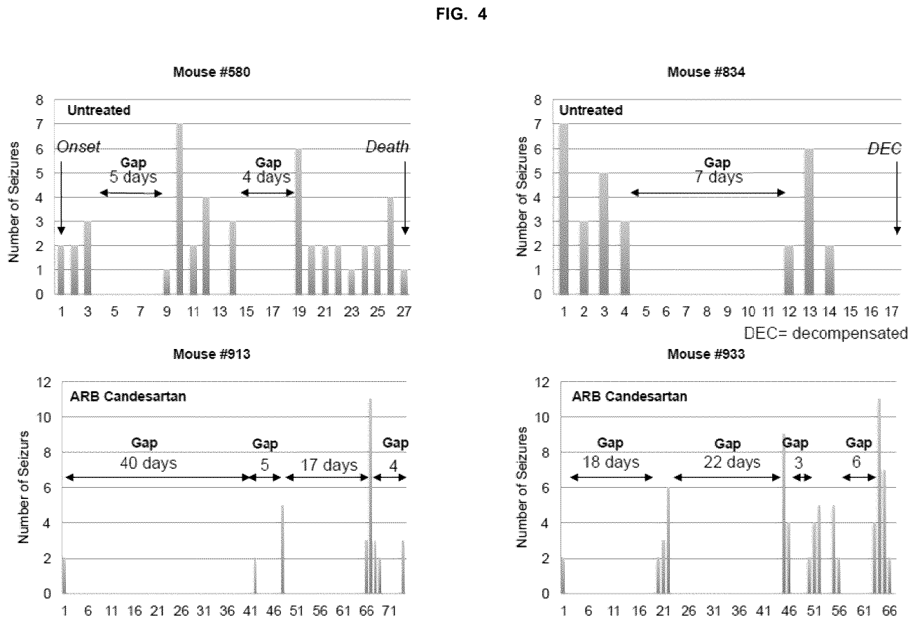

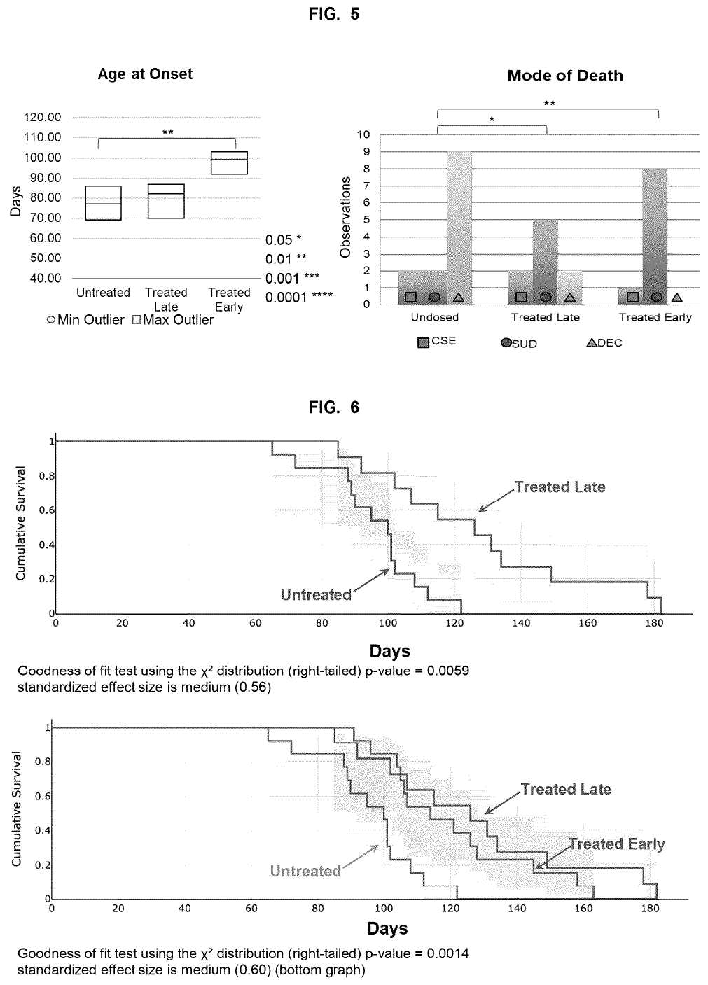

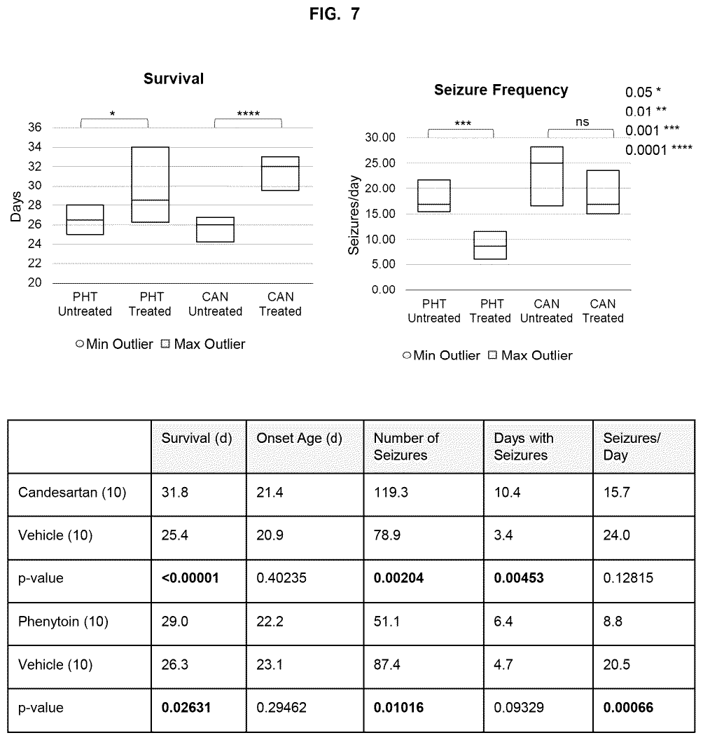

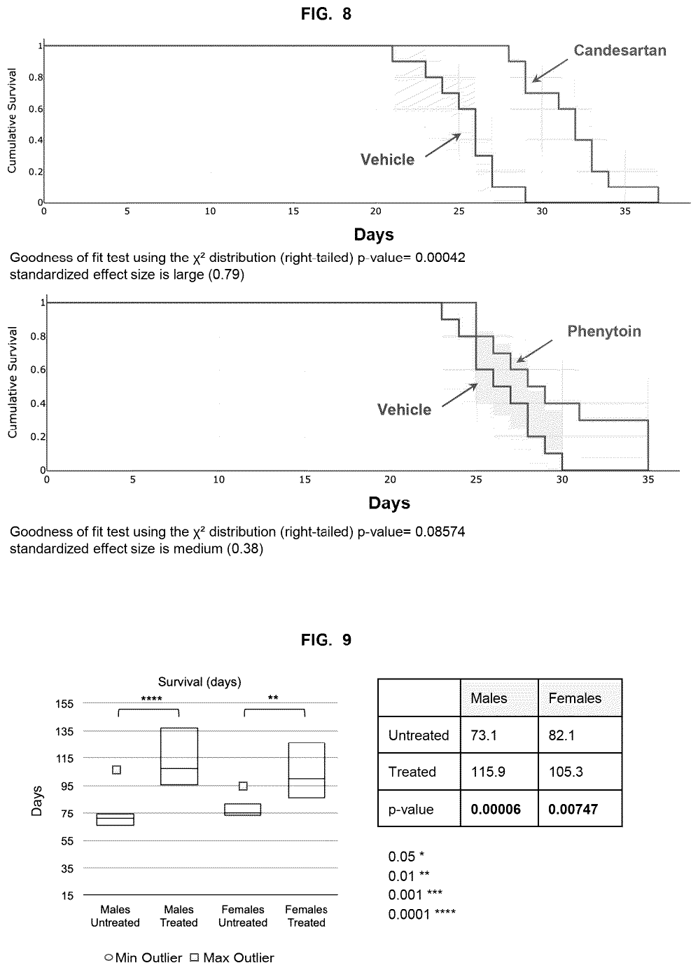

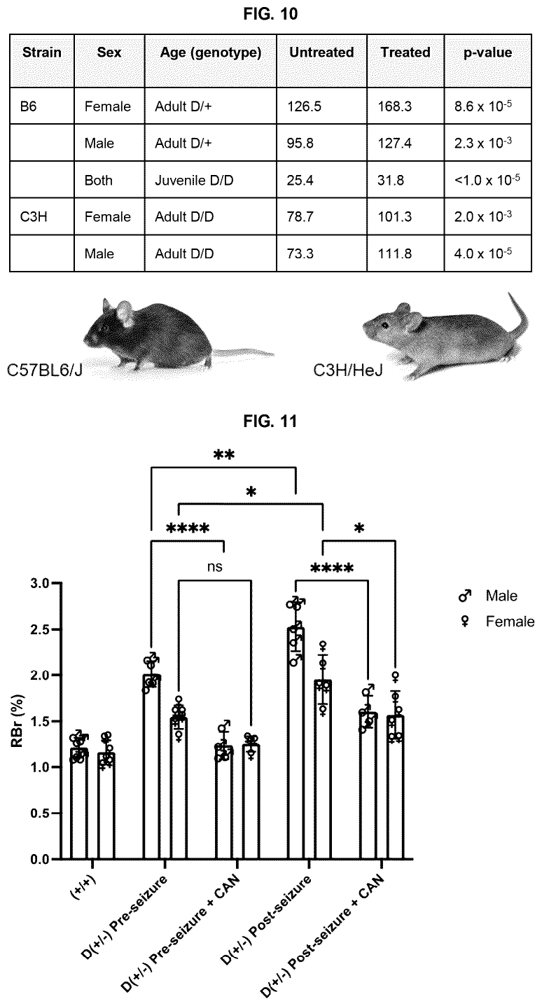

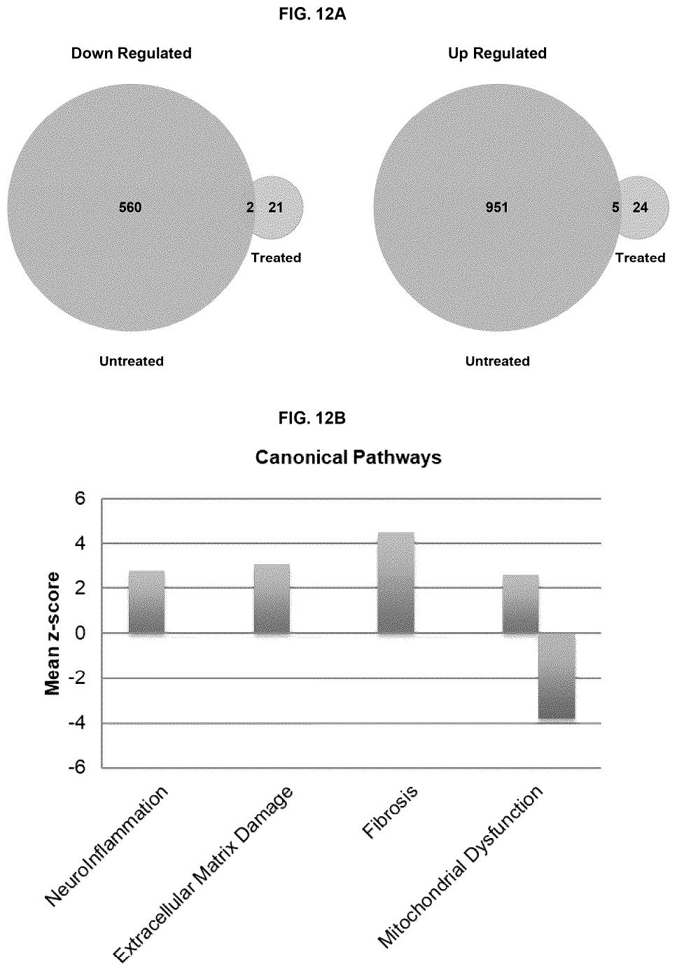

Referring now to FIGS. 1-12B, the present invention features compositions and methods for the treatment of epileptic disorders (e.g., epilepsy).

In some embodiments, the present invention features a method of preventing or treating an epileptic condition in a patient in need thereof. The method may comprise administering a therapeutically effective amount of an angiotensin receptor blocker (ARB) to the patient.

In some embodiments, the present invention may also feature a method of preventing or treating epilepsy in a patient in need thereof. The method may comprise administering a therapeutically effective amount of an angiotensin receptor blocker (ARB) to the patient.

In some embodiments, the present invention may also feature a method of protecting the blood-brain barrier (BBB) in a subject in need thereof. The method comprises administering a therapeutically effective amount of an angiotensin receptor blocker (ARB) to the subject. In some embodiments, the present invention features a method of maintaining blood-brain barrier function in a subject in need thereof; the method may comprise administering a therapeutically effective amount of an angiotensin receptor blocker (ARB) to the subject.

Without wishing to limit the present invention to any theories or mechanisms, it is believed that compositions (e.g., sartans; e.g., candesartan) that inhibit angiotensin II receptor type 1 (AT1R) and/or activate peroxisome proliferator-activated receptor-gamma (PPARγ) are efficacious in modifying and/or arresting the development of epilepsy (i.e., an epileptic disorder). Specifically, angiotensin receptor blockers (ARB; e.g., candesartan (CAN)) prevent or slow the progression of epileptogenesis through a multi-target mechanism involving genes affecting inflammatory-immune response pathways and those maintaining the integrity of the BBB.

In some embodiments, the present invention may also feature a method of preventing a seizure in a subject in need thereof, the method comprising administering a therapeutically effective amount of an angiotensin receptor blocker (ARB) to the subject. In some embodiments, the present invention may also feature a method of treating a disease that causes a seizure in a subject in need thereof by administering a therapeutically effective amount of an angiotensin receptor blocker (ARB) to the subject.

In some embodiments, the angiotensin receptor blocker (ARB) comprises a sartan or a derivative thereof. Non-limiting examples of sartans may include, but are not limited to candesartan, losartan, valsartan, irbesartan, telmisartan, eprosartan, azilsartan, olmesartan, or derivatives thereof. In some embodiments, the angiotensin receptor blocker (ARB) is candesartan. In other embodiments, the ARB may be azilsartan, eprosartan, irbesartan, losartan, olmesartan, telmisartan, or valsartan. Other angiotensin receptor blockers (ARBs; e.g., other sartans or derivatives thereof) may be used in accordance with the present invention.

As used herein, an “epileptic condition” may refer to a condition of the brain characterized by repeated seizures. A seizure is usually defined as a sudden alteration of behavior due to a temporary change in the electrical functioning of the brain. As used herein, “epilepsy” refers to a condition of recurrent, unprovoked seizures, but may include abnormal brain activity associated with higher risk of unprovoked seizures.

In some embodiments, the epileptic condition is pediatric epilepsy. In other embodiments, the epileptic condition is traumatic brain injury (TBI) or other neurodegenerative diseases (e.g., Huntington's Disease, Alzheimer's Disease, or Parkinson's Disease). In some embodiments, the epileptic condition may derive from a stroke. Other diseases that cause seizures, e.g., with age, may be prevented or treated with methods described herein.

The methods and/or treatments described herein may reduce seizure frequency in a patient. Additionally, the methods and/or treatments described herein may improve memory, learning, and alertness. Without wishing to limit the present invention to any theory or mechanism, it is believed that the use of the treatments herein improves the quality of life of the patient.

In some embodiments, the patient is a child. In some embodiments, the patient is an adult. Without wishing to limit the present invention to any theory or mechanism, treatments described herein may be used in a patient as young as one-years-old and may be beneficial in treating or preventing early child epilepsies.

In some embodiments, the ARB is delivered orally, e.g., in a pill or liquid form. In some embodiments, the ARB is delivered daily.

The present invention features a composition comprising an angiotensin receptor blocker (ARB) for use in a method of treating an epileptic condition in a patient in need thereof. In some embodiments, the present invention features a composition comprising an angiotensin receptor blocker (ARB) for use in a method of treating epilepsy in a patient in need thereof. In other embodiments, the present invention may also feature a composition comprising an angiotensin receptor blocker (ARB) for use in a method of treating a disease that causes a seizure in a patient in need thereof.

The present invention may also feature a composition comprising an angiotensin receptor blocker (ARB) for use in a method of preventing an epileptic condition in a patient in need thereof. In some embodiments, the present invention features a composition comprising an angiotensin receptor blocker (ARB) for use in a method of preventing epilepsy in a patient in need thereof. In other embodiments, the present invention may also feature a composition comprising an angiotensin receptor blocker (ARB) for use in a method of preventing a seizure in a patient in need thereof.

In some embodiments, the present invention features a composition comprising an angiotensin receptor blocker (ARB) for use in a method of protecting the blood-brain barrier (BBB) in a patient in need thereof. In some embodiments, the present invention features a composition comprising a sartan for use in a method of protecting the blood-brain barrier (BBB) in a patient in need thereof.

In some embodiments, the present invention may further feature a composition comprising a sartan for use in a method of preventing or treating an epileptic condition (e.g., epilepsy) in a patient in need thereof. In some embodiments, the present invention features a composition comprising a sartan for use in a method of preventing a seizure in a patient in need thereof. In other embodiments, the present invention features a composition comprising a sartan for use in a method of treating a disease that causes a seizure in a patient in need thereof.

In some embodiments, the presenting invention features the use of a compound comprising an angiotensin receptor blocker (ARB; e.g., a sartan, e.g., candesartan) in the manufacture of a medicament for the treatment of an epileptic condition (e.g., epilepsy).

### EXAMPLE

The following is a non-limiting example of the present invention. It is to be understood that said example is not intended to limit the present invention in any way. Equivalents or substitutes are within the scope of the present invention.

A healthy brain requires a healthy blood-brain barrier. The BBB controls the blood-to-brain exchange of nutrients, xenobiotics, blood components, and cells, ultimately maintaining the optimal brain milieu necessary for physiologic neuronal function. Disturbance of the blood-to-brain equilibrium can be a cause or consequence of central nervous system diseases, like epilepsy. Targeting of the damaged or dysfunctional BBB may represent a therapeutic approach to reduce seizure burden.

A mouse model with a knockin gain-of-function mutation (Scn8a-N1768D) in the voltage-gated sodium channel, NaV1.6 (encoded by SCN8A) leads to excess excitability in the brain and the production of seizures in virtually 100% of mice after an initial latent phase. Utilizing RNAseq of hippocampal tissue, different cellular and signaling pathways are altered in the latent phase, at the time of seizure onset, and during the chronic phase. Pathway analysis tools predicted that ARBs could potentially offset the effects of several of these pathological processes, including activation of peroxisome proliferator-activated receptor (PPAR) signaling and deactivation of Bone Morphogenetic Protein (BMP) and Transforming Growth Factor-β (TGF-β) signaling—pathways involved in regulating the BBB.

To determine whether blockade of the AT1 receptor with candesartan increased survival, mice were observed from an early age until the first tonic-clonic seizure (TC). Specifically, the aim was to determine whether candesartan increases the number of days mice lived after an initial tonic-clonic seizure (TC). Throughout the observation, the mice were orally given 4 mg/kg/day of candesartan via a peanut butter pellet.

Mice treated with candesartan (CAN) at different stages of epileptogenesis demonstrated statistically significant: 1) delay of age at seizure onset, 2) increased adult survival and a reduction in seizure frequency, and 3) increased juvenile survival.

Additionally, the effects of candesartan on the blood-brain barrier (BBB) and gene expression patterns both before and after seizure onset have been determined. Candesartan acts to prevent seizure onset and improve outcomes post-seizure, and it does so by protecting the BBB. Without wishing to limit the present invention to any theory or mechanism, it is believed that candesartan acts by a combined mechanism of action—both as an AT1R antagonist and a PPARγ (peroxisome proliferator-activated receptor-gamma) activator. These pathways are known to be involved in maintaining BBB function.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of treating an epileptic condition in a patient in need thereof, the method comprising administering a therapeutically effective amount of an angiotensin receptor blocker (ARB) to the patient.

2. A method of treating epilepsy in a patient in need thereof, the method comprising administering a therapeutically effective amount of an angiotensin receptor blocker (ARB) to the patient.

3. The method of claim 1 or claim 2, wherein the ARB comprises a sartan.

4. The method of any one of claims 1-3, wherein the ARB is candesartan.

5. The method of any one of claims 1-4, wherein the patient is a child.

6. The method of claim 5, wherein the epileptic condition is pediatric epilepsy.

7. The method of any one of claims 1-6, wherein the epileptic condition is traumatic brain injury (TBI).

8. The method of any one of claims 1-7, wherein the method reduces seizure frequency.

9. The method of any one of claims 1-8, wherein the ARB is administered orally.

10. The method of any one of claims 1-9, wherein the ARB is administered daily.

11. A method of preventing an epileptic condition in a patient in need thereof, the method comprising administering a therapeutically effective amount of an angiotensin receptor blocker (ARB) to the patient.

12. A method of preventing epilepsy in a patient in need thereof, the method comprising administering a therapeutically effective amount of an angiotensin receptor blocker (ARB) to the patient.

13. The method of claim 11 or claim 12, wherein the ARB comprises a sartan.

14. The method of any one of claims 11-13, wherein the ARB is candesartan.

15. The method of any one of claims 11-14, wherein the patient is a child.

16. The method of claim 15, wherein the epileptic condition is pediatric epilepsy.

17. The method of any one of claims 11-16, wherein the epileptic condition is traumatic brain injury (TBI).

18. The method of any one of claims 11-17, wherein the ARB is administered orally.

19. The method of any one of claims 11-18, wherein the ARB is administered daily.

20. A method of protecting the blood-brain barrier (BBB) in a subject in need thereof, the method comprising administering a therapeutically effective amount of an angiotensin receptor blocker (ARB) to the subject.

21. The method of claim 20, wherein the ARB comprises a sartan.

22. The method of claim 20 or claim 21, wherein the ARB is candesartan.

23. The method of any one of claims 20-22, wherein the subject is a child.

24. The method of claim 23, wherein the epileptic condition is pediatric epilepsy.

25. The method of any one of claims 20-24, wherein the epileptic condition is traumatic brain injury (TBI).

26. The method of any one of claims 20-25, wherein the method reduces seizure frequency.

27. The method of any one of claims 20-26, wherein the ARB is administered orally.

28. The method of any one of claims 20-27, wherein the ARB is administered daily.

29. A method of preventing a seizure in a subject in need thereof, the method comprising administering a therapeutically effective amount of an angiotensin receptor blocker (ARB) to the patient.

30. The method of claim 29, wherein the ARB comprises a sartan.

31. The method of claim 29 or claim 30, wherein the ARB is candesartan.

32. The method of any one of claims 29-31, wherein the subject is a child.

33. The method of any one of claims 29-32, wherein the ARB is administered orally.

34. The method of any one of claims 29-33, wherein the ARB is administered daily.

35. A composition comprising an angiotensin receptor blocker (ARB) for use in a method of treating an epileptic condition in a patient in need thereof.

36. A composition comprising an angiotensin receptor blocker (ARB) for use in a method of treating epilepsy in a patient in need thereof.

37. A composition comprising an angiotensin receptor blocker (ARB) for use in a method of protecting the blood-brain barrier (BBB) in a patient in need thereof.

38. A composition comprising an angiotensin receptor blocker (ARB) for use in a method of preventing a seizure in a patient in need thereof.

39. The composition of any one of claims 35-38, wherein the ARB comprises a sartan.

40. The composition of any of one of claims 35-39, wherein the ARB is candesartan.

41. The composition of any of one of claims 35-40, wherein the patient is a child.

42. The composition of claim 41, wherein the epileptic condition is pediatric epilepsy.

43. The composition of any of one of claims 35-42, wherein the epileptic condition is traumatic brain injury (TBI).

44. The composition of any of one of claims 35-43, wherein the composition reduces seizure frequency.

45. The composition of any of one of claims 35-44, wherein the ARB is administered orally.

46. The composition of any of one of claims 35-45, wherein the ARB is administered daily.

47. A composition comprising a sartan for use in a method of treating an epileptic condition in a patient in need thereof.

48. A composition comprising a sartan for use in a method of treating epilepsy in a patient in need thereof.

49. A composition comprising a sartan for use in a method of protecting the blood-brain barrier (BBB) in a patient in need thereof.

50. A composition comprising a sartan for use in a method of preventing a seizure in a patient in need thereof.
